(19) United States
(12) Patent Application Publication
(10) Pub. No.: US 2021/0401900 A1
(43) Pub. Date: Dec. 30, 2021

(54) ENTERIC COMPOSITION, FOOD OR BEVERAGE PRODUCT CONTAINING THE SAME, METHOD FOR CONTROLLING DISINTEGRATION TIME OF THE SAME, AND METHOD FOR MANUFACTURING THE SAME

(71) Applicants: Nutriomix INC, Pasadena, CA (US); NexgenNutrient Japan Co., Ltd, Osaka (JP)

(72) Inventors: Lijun Sun, La Canada Flintridge, CA (US); Takahito ICHI, Okayama (JP)

(21) Appl. No.: 16/768,895

(22) PCT Filed: Mar. 11, 2020
(86) PCT No.: PCT/JP2020/010474

§ 371 Date: Jun. 2, 2020

(30) Foreign Application Priority Data
Jan. 28, 2020 (JP) 2020-011408

(51) Int. Cl. A61K 35/742; A61K 9/107; A61K 47/44; A61K 47/46; A61K 47/42; A23L 33/135; A23L 33/115; A23P 10/40; A23P 20/18; A23L 33/185

(52) U.S. Cl. CPC A61K 35/742; A61K 9/107; A61K 47/44; A61K 47/46; A61K 47/42; A23V 2002/00; A23L 33/115; A23P 10/40; A23P 20/18; A23L 33/185; A23L 33/135

(57) ABSTRACT

PROBLEM TO BE SOLVED: There is provided an enteric composition that is suitable for addition to food or beverage products, such as yogurt, and has a disintegration time that is controllable to enable disintegration and content release in the small intestine or the large intestine, especially in the large intestine.

SOLUTION: An enteric composition includes a content and an enteric coating agent, the content being a mixture of probiotics and an edible hydrogenated oil emulsion, the content being coated with the enteric coating agent.

### TECHNICAL FIELD

The present invention relates to an enteric composition, a food or beverage product containing the same, a method for controlling the disintegration time of the same, and a method for producing the same.

### BACKGROUND ART

The intestinal tract of humans contains approximately 1000 bacterial species, and one hundred trillion to quadrillion bacteria reside in the small and large intestines. It has been known that these intestinal bacteria are deeply involved with nutrient metabolism, defense mechanisms, immune responses, and the like in human hosts.

Regarding the distribution of the intestinal bacteria, there are only a few resident bacteria in the duodenum and the upper portion of the small intestine, and the bacterial number increases along the small intestine from the upper to lower portion. In the region extending from the lower portion of the small intestine to the large intestine, due to slow passage of the intestinal content and almost oxygen-free environment, bacteria that prefer such an environment (anaerobic bacteria) are abundantly resident. In the large intestine, a far greater number of bacteria are found, and the bacterial composition is almost the same as the fecal bacterial flora. Large intestinal bacteria decompose undigested food from the small intestine.

The composition and balance of intestinal bacterial flora change with age. Additionally, the intestinal bacterial flora also varies depending on, for example, contents of meals, lifestyle habits, stress, constipation, medication intake, and diseases. For example, the intestinal bacteria are significantly changed by ingestion of a medical agent, such as an antibiotic and a proton pump inhibitor (PPI), and food poisoning.

Recently, it has become clear that the intestinal bacterial flora (intestinal flora) is closely related to diseases, such as obesity, diabetes, colorectal cancer, arteriosclerosis, and inflammatory bowel disease, and that the intestinal bacterial florae of patients affected by these diseases are significantly different from those of healthy people. The importance of the intestinal bacterial flora in maintaining good health in humans has been recognized more than ever before.

Therefore, maintaining or improving the balance of the intestinal bacterial flora to keep a healthy physiological homeostasis in the host can lead to prevention or treatment of diseases and is considerably important for the host to live long in good health.

Widely known medical agents to improve the intestinal bacterial flora of humans are live and active bacteria preparations that contain indigenous intestinal bacteria to regulate the intestinal environment. For example, a lactomin preparation (Streptococcus faecalis, Lactobacillus acidophilus), bifidobacteria, butyric acid bacteria (Miyairi strain), casei bacteria (Lactobacillus casei), and resistant lactic acid bacteria have been generally used.

However, oral ingestion of live probiotics results in their deactivation before they reach the large intestine due to the influence from digestive juices present in the stomach and the small intestine, such as gastric acid and bile acid. Such oral ingestion does not produce satisfactory results.

As a solution to the problem, an enteric preparation, which does not disintegrate in the stomach but disintegrates only after reaching the intestine, has been used. The enteric preparation is, for example, a capsule preparation designed to disintegrate in the small and large intestines to release an active ingredient (such as probiotics) encapsulated therein. Examples of the dosage form include fine granules and granules, in addition to capsules. The enteric preparation has a film that is not digestible in acidic gastric juice but is digestible in alkaline intestinal juices. This film provides the preparation with an acid-resistant property and an enteric function, thus enabling the delivery of live probiotics sensitive to gastric acid to the intestine. That is, the active ingredient is protected from gastric acid until reaching the small intestine or the large intestine, and the intact function of the encapsulated content can be provided in the small intestine or the large intestine.

As an enteric capsule containing probiotics, there has been reported a three-layer bifidobacterium powder-containing seamless capsule with a higher amount of bifidobacterium powder. The three-layer bifidobacterium powder-containing seamless capsule includes a content, an outer shell, and an outermost layer. The content includes bifidobacterium powder dispersed in an oil component. The content has a specific gravity of 1.0 to 1.4. The outer shell is a hydrogenated oil whose specific gravity is adjusted to 1.0 to 1.4 with a specific gravity regulator and encloses the content adjacently. The outermost layer is a monolayer made of a water-soluble film-forming agent. The bifidobacterium powder is contained in an amount of 40 to 60 wt % based on the weight of the content. The difference (Δd=dB−dA) between the specific gravity (dA) of the content and the specific gravity (dB) of the outer shell is within a range of −0.15 to +0.05 (Patent Literature 1).

As another example, there has been reported a large intestine delivery capsule preparation that is excellent in storage stability of encapsulated large intestinal beneficial bacteria and can deliver a sufficient amount of large intestinal beneficial bacteria in an active state to provide good effects on health. The preparation contains capsules each encapsulating large intestinal beneficial bacteria. Each capsule has a chitosan-containing layer and an enteric base material-containing layer in order from the inner side. The capsule also encapsulates sucrose fatty acid ester in an amount of 0.5 to 25 mass % relative to the whole amount of the content of the capsule (Patent Literature 2).

### CITATION LIST

### Patent Literature

Patent Literature 1: Japanese Patent No. 5989953
Patent Literature 2: JP-A-2017-149681

### SUMMARY OF INVENTION

### Technical Problem

However, conventional enteric capsule preparations, especially enteric capsules containing probiotics have a rather large size (at least 1 to 2 mm in diameter), whether the forms are hard capsules or seamless capsules, and therefore are difficult to appropriately disperse in food or beverage products. Moreover, in the manufacturing of food or beverage products containing such conventional enteric capsules, problems are likely to occur at the time of stirring ingredients, filling a container with the ingredients, and sealing the container.

Yogurt and other food or beverage products containing enteric capsules encapsulating lactic acid bacteria are present on the market, and in eating such food or beverage products, care needs to be taken to avoid chewing or crushing the enteric capsules. This is because they would otherwise be unable to deliver live lactic acid bacteria to the intestine. However, since enteric capsules have a rather large size as described above, it is uncomfortable to ingest them carefully without chewing.

Additionally, conventional enteric capsules often use a gelatinizing agent as the enteric coating agent, and those using such an enteric coating agent are usually sensitive to heat so that a capsule material is melted and destabilized by heat sterilization. Due to this disadvantage, heat sterilization cannot be performed on products using such conventional enteric capsules, for example, food or beverage products, and as a result, long-term storage of the products is difficult in some cases.

Enteric capsules disintegrate around pH 5.0. Generally, about 20% of them disintegrate in two hours after reaching the stomach, and the remainder reaches the small intestine (duodenum: pH 5.0) and disintegrates in two to three hours. Considering the retention time of food (stomach: two to four hours, small intestine: seven to nine hours, large intestine: 25 to 30 hours), enteric capsules are assumed to mostly disintegrate in the small intestine. As an inevitable result, the components encapsulated in enteric capsules to effectively work in the large intestine, like lactic acid bacteria and functional components, cannot sufficiently provide their actions. It is conceivable that lactic acid bacteria mostly have already been dead when reaching the large intestine.

Accordingly, an object of the present invention is to provide an enteric composition that is suitable for addition to food or beverage products, such as yogurt, and has a disintegration time that is controllable to enable disintegration and content release in the small intestine or the large intestine, especially in the large intestine.

### Solution to Problem

As a result of intensive studies to solve the problem, the inventors have found that the problem can be solved by uniformly dispersing probiotics in an edible hydrogenated oil composition used as a matrix and coating the mixture as a content with an enteric coating agent. Thus, the present invention has been completed.

The present invention is as follows.

[1] An enteric composition comprising a content and an enteric coating agent, the content being a mixture of probiotics and an edible hydrogenated oil emulsion, the content being coated with the enteric coating agent.
[2] The enteric composition according to [1], wherein the content further contains enzymes, a functional material, or a dietary fiber.
[3] The enteric composition according to [1] or [2], wherein the edible hydrogenated oil emulsion is an oil-in-water emulsion containing hydrogenated rapeseed oil, an emulsifier, and an aqueous solvent.
[4] The enteric composition according to any one of [1] to [3], wherein the enteric coating agent is zein, shellac, or hydroxypropyl methylcellulose (HPMC).
[5] The enteric composition according to any one of [1] to [4], wherein the content has a value (cfu/g) of the viable count of the probiotics (cfu)/the solid content of the edible hydrogenated oil emulsion (g) in a range of 1×10¹²/l to 1×10⁹/l.
[6] The enteric composition according to any one of [1] to [5], wherein the enteric composition is in the form of a powder with an average particle diameter of 1 μm to 1000 μm.
[7] The enteric composition according to any one of [1] to [6], wherein the enteric composition disintegrates in three to 30 hours after reaching the small intestine.
[8] A food or beverage product comprising the enteric composition according to any one of [1] to [6].
[9] The food or beverage product according to [8], wherein the food or beverage product is yogurt.
[10] A method for controlling the disintegration time of the enteric composition according to any one of [1] to [7], the method comprising adjusting the ratio between the viable count of the probiotics and the solid content of the edible hydrogenated oil emulsion.
[11] A method for producing the enteric composition according to [1], the method comprising: heating and stirring probiotics in water at 30 to 90° C. to prepare a probiotic liquid; adding a melted edible hydrogenated oil to an aqueous solution of an emulsifier with heating and stirring at 50 to 100° C. to prepare an edible hydrogenated oil emulsion; mixing the probiotic liquid with the edible hydrogenated oil emulsion, followed by drying and grinding the mixture into powder; and applying an enteric coating agent to the powder by spraying.

### Advantageous Effects of Invention

The enteric composition of the present invention has a disintegration time that is controllable as desired to enable the release of probiotics and functional components in the small and large intestines, especially in the large intestine, and to enable disintegration in a desired region of the intestine. Additionally, a powdery form of the enteric composition has a small particle size and is easy to ingest without chewing or crushing. Furthermore, in the manufacturing of yogurt and other food or beverage products containing such a powdered enteric composition, problems are less likely to occur at the time of stirring ingredients, filling a container with the ingredients and sealing the container. Moreover, unlike conventional enteric capsules, the enteric composition of the present invention does not contain a gelatinizing agent or the like, and thus food or beverage products containing the enteric composition can withstand heat sterilization and long-term storage.

### BRIEF DESCRIPTION OF DRAWINGS

FIG. 1 is a graph illustrating the results of Example 7.

### DESCRIPTION OF EMBODIMENTS

As described above, the enteric composition of the present invention includes a content that is a mixture of probiotics and an edible hydrogenated oil emulsion and an enteric coating agent that covers the content. Accordingly, the enteric composition is structurally different from conventional ones in which a content, such as probiotics, is covered by an edible hydrogenated oil-containing protective layer of, for example, a capsule.

The content includes probiotics and an edible hydrogenated oil emulsion as essential components, and additionally includes enzymes, a functional material, a dietary fiber, and the like as optional components. The enteric composition of the present invention is characterized in that the content is a mixture in which the probiotics are uniformly dispersed in the edible hydrogenated oil emulsion.

The probiotics refer to strains beneficial for the human body and means so-called helpful bacteria and good bacteria. Generally, lactic acid bacteria, bifidobacteria, and Bacillus subtilis (natto) are used for food or beverage products. One kind of probiotic or a combination of two or more kinds of probiotics can be used. These bacteria produce metabolites (organic acid, such as lactic acid and acetic acid) that maintain a mildly acidic environment in the intestine. Under the mildly acidic environment, the growth of acid-sensitive bad bacteria is reduced, the barrier function of the intestinal tract is enhanced, and such good effects on health can be expected. Particularly, in the enteric composition of the present invention intended for use in food or beverage products, a spore-forming lactic acid bacterium, Bacillus coagulans, which is a lactic acid bacterial species having high heat resistance, is preferred. Additionally, the form of the probiotics is preferably dried live bacteria powder in terms of dispersibility in the edible hydrogenated oil emulsion.

The edible hydrogenated oil emulsion is an oil-in-water emulsion in which an edible hydrogenated oil is emulsified and dispersed in an aqueous solvent. The edible hydrogenated oil emulsion at least contains an edible hydrogenated oil, an emulsifier, and an aqueous solvent (preferably water). As the edible hydrogenated oil, any edible one can be widely used. Examples of the edible hydrogenated oil include hydrogenated oil (in a solid state at approximately 60° C. or less), such as rapeseed oil, soybean oil, sesame oil, corn oil, cottonseed oil, rice oil, safflower oil, sunflower oil, olive oil, coconut oil, palm oil, and peanut oil. Hydrogenated oil blend of two or more kinds of oils may also be used. Among them, hydrogenated rapeseed oil is especially preferred.

The emulsifier used for the edible hydrogenated oil emulsion and its amount used are not limited as long as they do not interfere with the effects of the present invention. One kind of emulsifier alone or a combination of two or more kinds of emulsifiers may be used. Specifically, examples of the emulsifier include glycerin fatty acid ester, sucrose fatty acid ester, sorbitan fatty acid ester, lecithins, gum arabic, and processed starch. Among them, sucrose fatty acid ester is especially preferably used. The amount of the emulsifier used is generally 5 to 50 mass % relative to the edible hydrogenated oil.

The proportion of the solid content of the edible hydrogenated oil emulsion to the whole amount of the edible hydrogenated oil emulsion is from 10 to 50 mass % and preferably from 20 to 40 mass %. The proportion of less than 10 mass % cannot ensure an sufficient delay in the onset of disintegration, and the proportion of more than 50 mass % results in an increased viscosity and thus poor work efficiency.

As long as the effects of the present invention are obtained, any method may be used for preparing the edible hydrogenated oil emulsion. An example of the method will be given. First, 5 to 15 pts.mass of an emulsifier and 800 to 1200 pts.mass of water are mixed, and the mixture is heated at 80 to 100° C. for dissolution to give an aqueous solution of the emulsifier. While the aqueous solution is heated and stirred at 60 to 98° C., 40 to 80 pts.mass of a melted edible hydrogenated oil is added slowly. Afterwards, the mixture is emulsified by homogenization using a stirrer, such as a homomixer, thus giving the edible hydrogenated oil emulsion. As long as the effects of the present invention are not hindered, other components, for example, viscosity improver, such as xanthan gum, can be added to the edible hydrogenated oil emulsion.

In addition to the essential components, other components, for example, enzymes, a functional material, and a dietary fiber can be further included as the optional components in the content. In addition to various kinds of enzymes, enzyme cofactors, such as NAD, NADP, FMN, FAD, thiamine diphosphate, pyridoxal phosphate, coenzyme A, α-lipoic acid, and folic acid, are included in the “enzymes”.

The functional material is a material having a favorable effect on the human body in a broad sense, and the functional material for use as a food ingredient is also referred to as a functional food material or a functional material for food. Examples of the functional material include nucleic acid, collagen, peptide, chondroitin sulfate, insoluble dietary fiber, water-soluble dietary fiber, a gelatinizing agent, seaweed extract, and prebiotics. The prebiotics are generally defined as “indigestible food ingredients that promote the growth of good bacteria resident in the large intestine or inhibit the growth of harmful bacteria to bring beneficial effects on the host.” The “beneficial effects” include a wide-range of effects, such as infection prevention, immune response regulation, blood pressure-blood sugar regulation, promotion of mineral absorption, skin health, and stress reduction. Specific examples of the prebiotics include fructooligosaccharide, lactosucrose, theanderose, galacto-oligosaccharide, lactulose, isomalto-oligosaccharide, gentio-oligosaccharide, trehalose, xylo-oligosaccharide, soybean oligosaccharide, maltitol, lactitol, reduced isomaltulose, sorbitol, and xylitol. It is desirable to select prebiotics suitable to promote the growth of the selected probiotics.

The dietary fiber is categorized as a water-soluble dietary fiber, which dissolves in water, or an insoluble dietary fiber, which does not dissolve in water. Both types of dietary fibers have a high water absorption property, and when in contact with water or a solution, they take in the water or the solution in their structures to swell and become like a paste. Fermentation and decomposition of the dietary fiber in the large intestine helps the growth of intestinal bacteria, such as bifidobacteria, and the creation of favorable intestinal environment. For these reasons, the dietary fiber is preferably added. Examples of the water-soluble dietary fiber include pectin, enzymatic hydrolysates of guar bean, glucomannan, β-glucan, polydextrose, fructan, inulin, gum arabic, maltitol, psyllium, indigestible oligosaccharide, indigestible dextrin, agarose, sodium alginate, carrageenan, and fucoidan. Examples of the insoluble dietary fiber include cellulose, hemicellulose, lignin, chitin, and chitosan.

In the content, the value (cfu/g) of the viable count of the probiotics (cfu)/the solid content of the edible hydrogenated oil emulsion (g) is preferably in a range of 1×10¹²/l to 1×10⁹/l, and especially preferably in a range of 5×10¹¹/l to 1×10¹⁰/l. The reason is as follows. When the value (cfu/g) of the viable count of the probiotics (cfu)/the solid content of the edible hydrogenated oil emulsion (g) is in the above-stated range, the probiotics and the edible hydrogenated oil emulsion can be easily homogenized, and all the probiotic cells can be provided with a gentle coating with the edible hydrogenated oil emulsion. This allows the enteric composition to fulfill its purpose, which is to disintegrate over a large time range, three to 30 hours after reaching the small intestine, that is, release the probiotics in the large intestine. The gentle coating with the edible hydrogenated oil emulsion is presumed to contribute to reducing the probiotic mortality due to heating or the like, as well as providing the enteric coating.

Generally, probiotics, such as lactic acid bacteria, are less likely to colonize in the intestine, and therefore continuous supply of probiotics to the intestine for a certain period of time is desirable. However, most conventional enteric preparations disintegrate in two to three hours after reaching the small intestine (duodenum: pH 5.0). The retention time of food is two to four hours in the stomach, seven to nine hours in the small intestine, and 25 to 30 hours in the large intestine. This explains that conventional enteric preparations mostly disintegrate in the small intestine. However, lactic acid bacteria, functional components, and the like are considered to effectively work mainly in the large intestine, and for full effectiveness, delivering them to the large intestine is necessary. To this end, a possible approach is to control the amount of the enteric coating agent, but even so, enteric preparations will mostly disintegrate in two to three hours after reaching the small intestine. Therefore, this approach has a limitation, and it is usually difficult for enteric preparations to reach the large intestine. In contrast, the enteric composition of the present invention disintegrates over a time range of three to 30 hours after reaching the small intestine. This is because the disintegration is controlled by not only the enteric coating agent but also the edible hydrogenated oil composition. Therefore, the enteric composition of the present invention can deliver live lactic acid bacteria or the like to the large intestine. Controlling the intestinal retention time to enable the probiotics to reach the desired region is effective for probiotic colonization in the target region of the intestine and for improvement in the intestinal bacterial flora therein.

The enteric coating agent used to cover the content can be selected as appropriate from substances that have good solubility in an aqueous solution solvent having a neutral to alkaline pH value, which represents the intestinal environment, specifically, from substances that are soluble in a pH range of approximately 5 to 12.0. Examples of the enteric coating agent can include, zein, shellac, cellulose acetate phthalate, hydroxymethyl cellulose phthalate, hydroxypropylmethyl cellulose phthalate, hydroxypropyl methylcellulose acetate succinate, carboxymethyl cellulose, carboxymethyl ethyl cellulose, hydroxypropyl methylcellulose trimellitate, methacrylic acid copolymer, methacrylic acid-ethyl acrylate copolymer, methacrylic acid-methyl methacrylate copolymer, polyvinyl acetate phthalate, and polyvinyl butyrate phthalate. One of these substances alone or a combination of two or more of them may be used.

In the present invention, considering that the enteric composition of the present invention is used for addition to food or beverage products that need heat treatment, it is desirable not to use, as the enteric coating agent, a material that melts and becomes unstable during heat sterilization of the products, for example, a gelatinizing agent, such as gelatin, carrageenan, gellan gum, and alginic acid (or it is desirable to exclude such a material from the enteric coating agent).

Among the above-exemplified enteric coating agents, zein, shellac, and hydroxypropyl methylcellulose (HPMC) are preferred, and zein is particularly preferably used because it is unaffected by heat sterilization of the products. Zein is the main protein in corn. Zein is a group of prolamins, which are water-insoluble and 50 to 90% ethanol-soluble proteins, and does not refer to a single molecular species.

The enteric coating agent can contain an additive, such as a plasticizer, as desired. The plasticizer is not especially limited, and examples of the plasticizer can include triethyl citrate, propylene glycol, polyethylene glycol, and triacetin.

The amount of the enteric coating agent in the enteric composition of the present invention to the whole mass of the enteric composition is usually 0.5 to 20 mass % and preferably 5 to 10 mass %.

The enteric composition of the present invention can be formulated into various kinds of dosage forms, such as powders, fine granules, granules, and capsules, and powders are preferred in the case of addition to food or beverage products, especially yogurt. The powder preferably has an average particle diameter of 1 μm to 1000 μm, especially 5 μm to 100 μm for good dispersibility and high absorption in the body. The average particle diameter can be measured in a dispersion of the composition in ethanol using a laser diffraction scattering method.

The enteric composition of the present invention can be formulated into various kinds of dosage forms and used as it is as a solid preparation for internal use. Alternatively, the enteric composition can also be used as an additive for food or beverage products, pharmaceutical products, and quasi-drug products. The enteric composition is suitable for use as an ingredient for probiotic-containing functional foods, and can be added to food or beverage products, especially water-based food or beverage products, such as yogurt.

The enteric composition of the present invention can be prepared by, for example, the following procedure. Powdered probiotics are put in water and heated and stirred at 30 to 90° C. to prepare a probiotic liquid. The probiotic liquid and the edible hydrogenated oil emulsion prepared as described above are mixed with stirring for homogenization. Vacuum drying or freeze drying is performed, followed by grinding using a grinder, such as a pin mill and a hammer mill. To the ground product, an enteric coating agent is applied by spraying, and vacuum drying is performed, thus giving the enteric composition of the present invention.

In another aspect, the present invention provides a method for controlling the disintegration time of the enteric composition. The method includes adjusting a ratio between the viable count of the probiotics and the solid content of the edible hydrogenated oil emulsion. The larger the ratio of the viable count of the probiotics to the solid content of the edible hydrogenated oil emulsion becomes, the shorter the disintegration time of the enteric composition becomes. Specifically, the value of the ratio (cfu/g) of the viable count of the probiotics (cfu)/the solid content of the edible hydrogenated oil emulsion (g) is preferably adjusted to the range given above in the description of the enteric composition.

### EXAMPLES

Hereinafter, illustrative examples and comparative examples are shown to further clarify the effects of the present invention, but these are for illustrative purposes only and do not limit the present invention.

### Example 1

First, as an emulsifier, 10 g of sucrose fatty acid ester (sucrose stearic acid ester S-1570 manufactured by Mitsubishi-Chemical Foods Corporation) and 913 ml of water were heated and stirred at 90° C. and 300 rpm for 20 minutes, and while the aqueous solution was stirred, 75 g of melted hydrogenated rapeseed oil was added slowly. Next, the resultant was homogenized at 80° C. and 8000 rpm for five minutes and 2 g of xanthan gum was added to prepare an edible hydrogenated oil emulsified liquid.

Meanwhile, 200 ml of water and 36 g of spore-forming lactic acid bacteria (Bacillus coagulans, Lactospore (registered trademark)) as probiotics (viable bacteria count: 2.7×10¹⁰ cfu/g) were heated and stirred at 40° C. and 300 rpm for 20 minutes to prepare a probiotic liquid.

413 g of the edible hydrogenated oil emulsified liquid (solid content: 18 g) and 236 g of the probiotic liquid (solid content: 36 g) were mixed at 40° C., vacuum drying was performed at 30° C. for 24 hours, and the obtained dried product was ground with a grinding mill for 10 seconds (100 mesh pass: 90% or more).

On the surface of the dried ground product thus obtained, 180 ml (solid content: 18 g) of a zein solution (10% solution) as an enteric coating agent prepared by dissolving zein in 80% ethanol was applied three times by spraying. Afterwards, vacuum drying was performed at 20° C. for 24 hours to obtain a powder (1.35×10¹⁰ cfu/g) with an average particle diameter of 20 μm (Example Product 1). The value of 1.35×10¹⁰ cfu/g is a calculated value based on the mass ratio of the solid contents between the probiotic powder, the edible hydrogenated oil emulsion, and the coating agent of Example Product 1, which was 2:1:1.

### Example 2

To examine the survival rate of the lactic acid bacteria in Example Product 1, the lactic acid bacteria count was measured by the BCP plate count agar method. In order to kill other bacteria, fungi, and yeast, BCP plate count agar was heated at 75° C. for 30 minutes prior to inoculation. Next, 24.6 g of the medium was dissolved in 1000 ml of warm purified water, followed by sterilization at 121° C. for 15 minutes. While kept at approximately 50° C., the medium was mixed with a certain amount of Example Product 1. Cultivation was performed at 35 to 37° C. for 72 hours. Then, the lactic acid bacteria count (cfu/g) was calculated from the number of colonies (each colony was surrounded by a yellow halo) appeared in the cultivation and used for evaluation. For comparison, the calculated value of Example Product 1 was used. The results are shown in Table 1. As seen from Table 1, the high survival rate of the probiotics was confirmed in Example Product 1, which was obtained by the above-described production method.

TABLE 1

| | Bacteria count | Survival rate |
|---|---|---|
| | (cfu/g) | (%) |
| Example Product 1 | 1.35 × 10¹⁰ | 100% |
| (calculated value) | | |
| Example Product 1 | 1.3 × 10¹⁰ | 96.30% |
| (measured value) | | |

The measured value of the viable bacteria count in the probiotic raw material: 2.7×10¹⁰ (cfu/g)

### Example 3

To examine the survival rate of the probiotics in a yogurt containing Example Product 1, in other words, to examine the preservability of the probiotics in the powder of Example Product 1 in the yogurt, the probiotic count in the yogurt was measured over time by the BCP plate count agar method. Cultivation was performed at 35° C. for 72 hours. The probiotic count (cfu/g) was calculated from the number of colonies appeared in the cultivation and used for evaluation. The yogurt used here was a sterilized yogurt (MOMCHLOVTSI, manufactured by Bright Dairy & Food Co., Ltd. (China)), in which most lactic acid bacteria were assumed dead. To 200 g of the yogurt, the powder of Example Product 1 was added at 2 mass %, an acceleration test was conducted at a temperature of 40° C., and the probiotic count was measured over time until after an elapse of 60 days from immediately after the addition. At each time point for the measurement of the probiotics, sterile water was added to the stored yogurt, and the Example Product 1 contained in the yogurt was collected by centrifugation (3000 rpm, 20 minutes) and used as a measurement sample. Table 2 shows the results. As seen from Table 2, the high survival rate of the lactic acid bacteria was confirmed in the yogurt containing Example Product 1.

TABLE 2

| | Probiotic | Survival | |
|---|---|---|---|
| | count | rate | |
| | (cfu/g) | (%) | Characteristics |
| At start | 1.35 × 10¹⁰ | 100% | No change |
| (added at 2%) | | | |
| 10 days later | 1.25 × 10¹⁰ | 93% | No change |
| 20 days later | 1.05 × 10¹⁰ | 78% | Particles with an absorbed |
| | | | water content of about 5% |
| | | | were observed |
| 30 days later | 0.9 × 10¹⁰ | 67% | Particles with an absorbed |
| | | | water content of about 5% |
| | | | were observed |
| 60 days later | 0.85 × 10¹⁰ | 63% | Particles with an absorbed |
| | | | water content of about 10% |
| | | | were observed |

### Examples 4 to 6, Comparative Example 1

The main purpose of the enteric composition is to enable probiotic colonization in the small intestine and the large intestine. Accordingly, the enteric composition is intended to have a disintegration time that is controllable to enable disintegration and content release in the small intestine or the large intestine, especially in the large intestine. To examine the disintegration time of Example Product 1, carminic acid was used. Carminic acid is a dye with high heat resistance and high acid resistance and is easy to use for disintegration property evaluation. Except that carminic acid was added to the edible hydrogenated oil emulsified liquid, and that the amounts indicated in Table 3 were used for zein, the edible hydrogenated oil emulsified liquid, carminic acid, and the probiotics, the same procedure as in Example 1 was performed to prepare powders (Example Products 2 to 4, Comparative Product 1). In Table 3, the concentrations of zein and the emulsified liquid in each obtained powder indicate the proportion of the solid content of zein and the proportion of the solid content of the emulsified liquid to the sum of the solid content of the emulsified liquid and the amount of the probiotics.

TABLE 3

| | | Amount of | Amount of | | Amount of | | |
|---|---|---|---|---|---|---|---|
| | | zein | emulsified liquid | | cochineal | Probiotics | |
| | Conditions | (g) | (g) | | (g) | (g) | |
| Comparative Product 1 | Zein 6% | 30 g (3 g) | 0 | | 0.1 g | 50 | g |
| Example Product 2 | Zein 6% + | 30 g (3 g) | 28.4 g | (2.5 g) | 0.1 g | 47.5 | g |
| | emulsified liquid 5% | | | | | | |
| Example Product 3 | Zein 6% + | 30 g (3 g) | 142 g | (12.5 g) | 0.1 g | 37.5 | g |
| | emulsified liquid 25% | | | | | | |
| Example Product 4 | Zein 6% + | 30 g (3 g) | 284 g | (25 g) | 0.1 g | 25 | g |
| | emulsified liquid 50% | | | | | | |

(1) The amount of zein: each value in the parentheses indicates the solid content. 10% zein solution.
(2) The amount of emulsified liquid: each value in the parentheses indicates the solid content. The percentage of the solid content in the emulsified liquid is 8.7%.

### Example 7

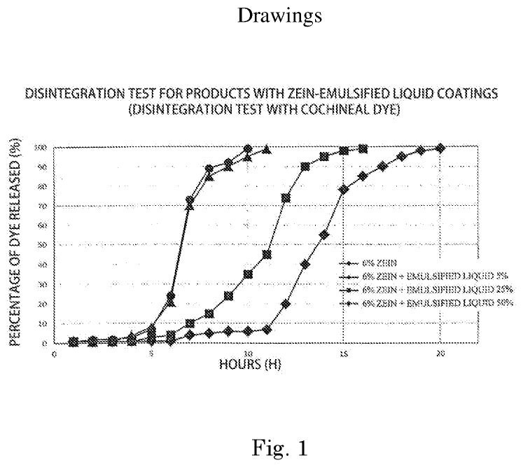

5 g of each of the powders of Example Products 2 to 4 and Comparative Product 1 was suspended in a pseudo-intestinal solution (pH 6.8, McIlvaine buffer solution (citrate-phosphate buffer solution)). The suspension was sampled at every hour to measure the absorbance of cochineal dye (buffer solution at pH 3.0 was used) and determine the percentage of the dye released. FIG. 1 illustrates the results.

It has been found from the results of FIG. 1 that Example Products 2 to 4, in which the edible hydrogenated oil emulsified liquid was mixed with the probiotics (lactic acid bacteria), showed a delayed onset of disintegration. Also shown was a tendency that the disintegration time in the intestine lengthens as the proportion of the solid content of the emulsified liquid increases.

1. An enteric composition comprising a content and an enteric coating agent, the content being a mixture of probiotics and an edible hydrogenated oil emulsion, the content being coated with the enteric coating agent.

2. The enteric composition according to claim 1, wherein
the content further contains enzymes, a functional material, or a dietary fiber.

3. The enteric composition according to claim 1, wherein
the edible hydrogenated oil emulsion is an oil-in-water emulsion containing hydrogenated rapeseed oil, an emulsifier, and an aqueous solvent.

4. The enteric composition according to claim 1, wherein
the enteric coating agent is zein, shellac, or hydroxypropyl methylcellulose (HPMC).

5. The enteric composition according to claim 1, wherein
the content has a value (cfu/g) of the viable count of the probiotics (cfu)/the solid content of the edible hydrogenated oil emulsion (g) in a range of 1×10¹²/l to 1×10⁹/l.

6. The enteric composition according to claim 1, wherein
the enteric composition is in the form of a powder with an average particle diameter of 1 μm to 1000 μm.

7. The enteric composition according to claim 1, wherein
the enteric composition disintegrates in three to 30 hours after reaching the small intestine.

8. A food or beverage product comprising an enteric composition comprising a content and an enteric coating agent, the content being a mixture of probiotics and an edible hydrogenated oil emulsion, the content being coated with the enteric coating agent.

9. The food or beverage product according to claim 8, wherein
the food or beverage product is yogurt.

10. A method for controlling the disintegration time of an enteric composition comprising a content and an enteric coating agent, the content being a mixture of probiotics and an edible hydrogenated oil emulsion, the content being coated with the enteric coating agent, the method comprising
adjusting a ratio between the count of the probiotics and the solid content of the edible hydrogenated oil emulsion.

11. A method for producing the enteric composition according to claim 1, the method comprising:
heating and stirring probiotics in water at 30 to 90° C. to prepare a probiotic liquid;
adding a melted edible hydrogenated oil to an aqueous solution of an emulsifier with heating and stirring at 50 to 100° C. to prepare an edible hydrogenated oil emulsion;
mixing the probiotic liquid with the edible hydrogenated oil emulsion, followed by drying and grinding the mixture into powder; and
applying an enteric coating agent to the powder by spraying.
